(19) United States
(12) Patent Application Publication
(10) Pub. No.: US 2026/0335499 A1
(43) Pub. Date: Oct. 29, 2026

(54) PECTIN FILMS

(71) Applicant: The Brigham and Women's Hospital, Inc., Boston, MA (US)

(72) Inventor: Steven J. Mentzer, Boston, MA (US)

(21) Appl. No.: 19/113,609

(22) PCT Filed: Sep. 21, 2023
(86) PCT No.: PCT/US2023/074784

§ 371 Date: Mar. 20, 2025

Related U.S. Application Data
(60) Provisional application No. 63/376,754, filed on Sep. 22, 2022.

(51) Int. Cl. A61L 24/08; A61L 24/00; A61L 24/06

(52) U.S. Cl. CPC A61L 24/08; A61L 24/0015; A61L 24/06

(57) ABSTRACT

The present disclosure describes a bioadhesive, pectin-based polymer film including: a bioadhesive, pectin-based polymer film including: a high-methoxyl pectin (HMP); tannic acid; and water, wherein the film has a controlled degradation rate that is dependent upon the amount of tannic acid present in the film. Methods of preparing the bioadhesive polymer pectin-based polymer films and methods of treating a wound of a subject in need thereof are also described.

### CLAIM OF PRIORITY

This application claims the benefit of U.S. Provisional Patent Application Ser. No. 63/376,754 filed on Sep. 22, 2022, the entire contents of which are hereby incorporated by reference.

### TECHNICAL FIELD

The present disclosure describes bioadhesive polymer compositions including one or more films comprising a polymer comprising high methoxyl pectin (HMP), tannic acid, and water. The disclosure also describes methods of treating a wound in a subject in need thereof with these films and methods of preparing and testing the films.

### BACKGROUND

The mesothelium is a membrane composed of simple squamous epithelial cells of mesodermal origin, which forms the lining of several body cavities: the pleura (pleural cavity around the lungs), peritoneum (abdominopelvic cavity including the mesentery, omenta, falciform ligament, and the perimetrium), and pericardium (fluid-filled sac surrounding the heart). Injury to the mesothelium creates a problem, not simply because of the compromised barrier function, but because the movement and forces associated with visceral organs (e.g., lung, bowel, and heart) can significantly compromise mesothelial healing. The slippery or non-adhesive surface of the mesothelium makes the use of traditional adhesives or sealants difficult. Thus, improvements in the design of bioadhesives are continually sought.

### SUMMARY

Certain aspects of the present disclosure are directed to bioadhesive, pectin-based polymer composition and method of manufacture and use comprising any feature described herein, either individually or in combination with any feature, in any configuration.

Certain aspects of the present disclosure are directed to bioadhesive, pectin-based polymer films including a high-methoxyl pectin (HMP), tannic acid, and water. In some embodiments, the film has a controlled degradation rate that is dependent upon the amount of tannic acid present in the film.

In some embodiments, HMP is present at an initial concentration of about 1% w/w to about 10% w/w.

In some embodiments, HMP is present at an initial concentration of about 3% w/w.

In some embodiments, the tannic acid is present at an initial concentration of 0.2% w/w to about 2% w/w.

In some embodiments, the water is present at an initial concentration of about 1% w/w to about 10% w/w.

In some embodiments, the water is present at an initial concentration of about 5% w/w.

In some embodiments, the film has a degradation half-life ranging from about 20 hours to about 200 hours.

In some embodiments, the degradation is facial erosion.

In some embodiments, the film has a thickness of about 40 to 3000 microns.

In some embodiments, the film has a thickness of about 40 to 50 microns.

In some embodiments, the film is a moldable film.

In some embodiments, the film comprises one or more active agents.

In some embodiments, the one or more active agents comprise an anti-thrombotic agent, an anti-inflammatory agent, a hormone, a cytokine, an osteogenic factor, a chemotactic factor, a protein or peptide that contain an arginine-glycine-aspartate (“RGD”) motif, an analgesic, an anesthetic, a vasoconstrictor, a clotting factor, a chemotherapy agent, an immunotherapy agent, a growth factor, or any combination of distinct active agents thereof.

In some embodiments, the anti-thrombotic agent is heparin, tissue plasminogen activator (tPA), or a combination thereof.

In some embodiments, the anti-inflammatory agent is aspirin, ibuprofen, ketoprofen, a non-steroidal anti-inflammatory drug, or a combination of distinct anti-inflammatory agents thereof.

In some embodiments, the vasoconstrictor is norepinephrine, epinephrine, phenylpropanolamine, dopamine, metaraminol, methoxamine, ephedrine, propylhexedrine, fibrillar collagen, thrombin, fibrin, or a combination thereof.

In some embodiments, the growth factor comprises transforming growth factor alpha (TGF-α), TGF-β, tumor necrosis factor-alpha (TNF-α), vascular endothelial growth factor (VEGF), leukemia inhibitory factor (LIF), interleukins, IL-1 through IL-7, colony-stimulating factors, macrophage colony-stimulating factor (m-CSF), granulocyte colony-stimulating factor (G-CSF), granulocyte macrophage colony-stimulating factor (GM-CSF), fibroblast growth factor (FGF), epidermal growth factor (EGF), insulin-like growth factor, connective tissue growth factor (CTGF), hepatocyte growth factor (HGF), angiopoietin-1-4, platelet-derived growth factor (PDGF), or any combination of distinct growth factors thereof.

In some embodiments, the HMP is a citrus HMP having a degree of methoxylation greater than about 50%.

In some embodiments, the bioadhesive, pectin-based polymer film binds to a mesothelial tissue.

In some embodiments, the mesothelial tissue comprises a mesothelial tissue from a visceral organ.

Another aspect of the present disclosure is directed to a method of covering and sealing a wound in an injured mesothelial tissue of a subject in need thereof, the method including: providing a bioadhesive pectin-based polymer film comprising: a high-methoxyl pectin (HMP), tannic acid, and water, contacting the wound with the film, and applying pressure to the film, thereby sealing the wound in the injured mesothelial tissue of the subject. In some embodiments, the film has a controlled degradation rate that is dependent upon the amount of tannic acid present in the film.

In some embodiments, the HMP is present at an initial concentration of about 1% w/w to about 10% w/w.

In some embodiments, the HMP is present at an initial concentration of about 3% w/w.

In some embodiments, the tannic acid is present at an initial concentration of about 0.2% w/w to about 2% w/w.

In some embodiments, the water is present at an initial concentration of about 1% w/w to about 10% w/w.

In some embodiments, the water is present at an initial concentration of about 5% w/w.

In some embodiments, the film has a degradation half-life ranging from about 20 hours to about 200 hours.

In some embodiments, the degradation is facial erosion.

In some embodiments, the film has a thickness of about 40 to 200 microns.

In some embodiments, the film comprises one or more active agents.

In some embodiments, the one or more active agents comprise an anti-thrombotic agent, an anti-inflammatory agent, a hormone, a cytokine, an osteogenic factor, a chemotactic factor, a protein or peptide that contain an arginine-glycine-aspartate (“RGD”) motif, an analgesic, an anesthetic, a vasoconstrictor, a clotting factor, a chemotherapy agent, an immunotherapy agent, a growth factor, or any combination of distinct active agents thereof.

In some embodiments, the HMP is a citrus HMP having a degree of methoxylation greater than about 50%.

In some embodiments, the injured mesothelial tissue comprises a mesothelial tissue from a visceral organ.

The terms “subject” or “patient” as used herein refer to any mammal (e.g., a human or a veterinary subject, e.g., a dog, cat, horse, cow, goat, sheep, mouse, rat, or rabbit) to which a composition or method of the present disclosure may be administered, e.g., for experimental, diagnostic, prophylactic, and/or therapeutic purposes. The subject may seek or need treatment, require treatment, is receiving treatment, will receive treatment, or is under care by a trained professional for a particular disease or condition.

As used in the specification and the appended claims, the singular forms “a,” “an” and “the” include plural referents unless the context clearly dictates otherwise.

As used herein, the term “active agent” is any molecule which is encapsulated, conjugated, fused, dispersed, embedded, mixed, or otherwise affixed to any of the film compositions described herein and is useful for a disease therapy.

As used herein, the expression “pharmaceutically acceptable” applies to a composition which contains composition ingredients that are compatible with other ingredients of the composition as well as physiologically acceptable to the recipient (e.g., a mammal such as a human) without the resulting production of excessive undesirable and unacceptable physiological effects or a deleterious impact on the mammal being administered the pharmaceutical composition. In some embodiments, a composition for use comprises one or more carriers, useful excipients, and/or diluents.

As used herein, the term “biodegradable” refers to a substance which may be broken down by microorganisms, or which spontaneously breaks down over a relatively short time (within about 14 days to about 6 months) when exposed to environmental conditions commonly found in a physiological environment. For example, the compositions described herein may be degraded by enzymes which are present in the body (e.g., the mesothelial environment).

As used herein, the term “bioadhesive” refers to a composition that can securely bind to living tissue.

As used herein, the term “biocompatible” means a composition that is physiologically acceptable to a living tissue and organism.

As used herein, a “pectin” is any one of a family of galacturonic acid-rich polysaccharides including homogalacturonan, rhamnogalacturonan I, and the substituted galacturonans rhamnogalacturonan II (RG-II) and xylogalacturonan (XGA), as described in Mohnen, “Pectin Structure and Biosynthesis,” Current Opinions in Plant Biology, 11:266-277, 2008. High methoxyl pectins and amidated pectins are variations of the pectin family. As used herein, the terms “high-methoxyl pectin,” “high-methyl pectin,” and high methyl ester-pectin” are used interchangeably.

Ranges may be expressed herein as from “about” one particular value and/or to “about” another particular value. When such a range is expressed, another embodiment includes from the one particular value and/or to the other particular value. The use of the term “about,” as used herein, refers to an amount that is near the stated amount by about 10% including increments therein. For example, “about” can mean a range including the particular value and ranging from 10% below that particular value and spanning to 10% above that particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Where values are described in the present disclosure in terms of ranges, endpoints are included. Furthermore, it should be understood that the description includes the disclosure of all possible sub-ranges within such ranges, as well as specific numerical values that fall within such ranges irrespective of whether a specific numerical value or specific sub-range is expressly stated.

Other features and advantages of the present disclosure will be apparent from the following detailed description and figures, and from the claims.

Various embodiments of the features of this disclosure are described herein. However, it should be understood that such embodiments are provided merely by way of example, and numerous variations, changes, and substitutions can occur according to those skilled in the art without departing from the scope of this disclosure. It should also be understood that various alternatives to the specific embodiments described herein are also within the scope of this disclosure.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features and advantages of the invention will be apparent from the following detailed description and figures, and from the claims.

### DESCRIPTION OF DRAWINGS

FIGS. 1A-1B are schematic illustrations of the different modes of mass loss from degradable polymers. FIG. 1A is a schematic illustration showing surface erosion, bulk erosion, and facial erosion. The conventional understanding of polymer degradation includes surface erosion and bulk erosion. Facial erosion describes the degradation of polymers adherent to visceral organ surfaces. FIG. 1B is a schematic illustration showing an example degradation circular pectin films immersed in water. The pectin films demonstrate progressive swelling and dissolution.

FIGS. 2A-2D are graphs characterizing the fluorescein fluorescence and trypan blue quenching. FIG. 2A shows the fluorescein standard curve was linear at lower concentrations. Modest nonlinearity at the higher concentrations was likely due to auto-quenching and detector saturation. FIG. 2B shows Trypan blue was used to quench the released fluorescein tracer. The effectiveness of trypan blue quenching was assessed over a 2-fold range of fluorescein concentrations from 1.95 μg/mL (light gray line) to 125 μg/mL (black line). At these fluorescein concentrations, the experimentally obtained Stern-Vollmer plot was linear with an aggregate constant of 7.15×10⁵ M⁻¹ (R²=0.9754). FIG. 2C shows the quenching of 125 μg/mL of fluorescein (circles) was assessed at increasing volumes of trypan blue (squares) by using standard 0.4% trypan blue solutions. Greater than 90% of the detectable fluorescence was reduced above 5 μL of trypan blue. FIG. 2D shows the trypan blue-associated quenching was rapid and stable. Maximal quenching occurred within seconds of the addition of the trypan blue at all volumes from 2 μL (light gray line) to 17 μL (black line). Error bars=1 standard deviation (SD).

FIGS. 3A-3B are graphs quantifying the fluorescein tracer embedded into the pectin films. FIG. 3A shows that pectin films embedded with fluorescein demonstrated a mostly linear relationship to fluorescein concentration (solid line). A slight increase in fluorescence was noted after pectin dissolution with distilled water (10 μL) (dotted line). FIG. 3B shows that tracer fluorescence, at a variety of concentrations, was stable for 72 h. Serial dilutions are shown from 37 μg/mL (black line) to 2.3 μg/mL (light gray line). Error bars=1 SD.

FIGS. 4A-4B show the empirical evaluation of a variety of laboratory materials for matrix characteristics suitable for evaluating facial erosion. FIG. 4A shows the surface area of the superficial matrix (Surface Area-1) compared to the surface area of the deeper layer (Surface Area-2). A variety of commercial and laboratory porous matrices typically used as filters were screened. Double-thickness matrices were used to evaluate lateral and penetrating diffusion of the trypan blue perfusate. Commercial filters with pore sizes between 40-50 μm resulted in the greatest surface and penetrating diffusion (a). Laboratory filters, generally composed of nitrocellulose and cellulose acetate, had pore sizes between 6 and 10 μm. These filters had comparable surface diffusion, but significantly less penetrating diffusion (FIG. 4A (b)) than the larger pore matrices (FIG. 4A (a)). Specialty filters, composed of synthetic microfibers, nylon membranes, and biologic matrices (e.g., collagen), demonstrated limited trypan blue diffusion (FIG. 4A (c)). FIG. 4B shows how after initial application of the trypan blue (TB), the optical properties of the matrices were stable over 40 min. A notable exception was the fluorescence measured at low concentrations of trypan blue (arrow). Because of the potential reflectance artifact from the white matrices, a black 112 mm²×160 um matrix with 6 μm-pore size was used for subsequent experiments (CAT#: 4740C10, Thomas Scientific). Error bars=1 SD.

FIGS. 5A-5D area schematic illustrations of a facial erosion assay. FIG. 5A illustrates pectin with fluorescein tracer embedded in pectin free volume. FIG. 5B illustrates trypan blue loaded into cellulose-based 6 μm-pore filter matrix (12 mm diameter) providing the interface. FIG. 5C illustrates the contact of the trypan blue matrix and the fluorescein-labeled pectin results in gradual loss of fluorescein from the pectin matrix. Loss of the fluorescein is quantitatively assessed by the microfluorimeter from the bottom of the plate (bottom arrow). FIG. 5D illustrates a microfluidics syringe pump perfusion system that allowed for the computer-controlled parallel perfusion of 8 samples. Most samples were perfused at 1 μL/h.

FIGS. 6A-6B are graphs quantifying the dose-dependent effect of tannic acid on pectin film degradation over 3 days of continuous microperfusion. FIG. 6A shows substantial pectin facial erosion occurs over the first day (light circles). FIG. 6B shows surface erosion demonstrated reproducible kinetics. Plots of 1/RFU demonstrated straight lines for all samples (R² ranged from 0.9943 to 0.9332). Facial erosion half-life for pectin alone was estimated at 14 h. The estimated half-life of pectin increased with increasing tannic acid concentration: 0.25%, 20 h; 0.75%, 69 h; 1.25%, 79 h; 1.5%, 99 h; and 1.75%, 154 h.

FIGS. 7A-7D are graphs showing the effect of tannic acid on the physical properties of citrus pectin. Three pectin variants-citrus, soybean and potato pectins-were analyzed for the effect of tannic acid on their physical properties in the glass phase. FIG. 7A is a graph quantifying the burst strength of citrus, soybean and potato pectins. The standard burst testing was performed with a 5 mm stainless steel ball, which impacted the films at 2 mm/sec with simultaneous distance and force recordings. All films were studied at 5% water content (citrus 5.1±0.8, soybean 5.2±0.2 and potato 5.4±0.4). An intermediate concentration of tannic acid (0.75%) was chosen for detailed testing. Citrus pectin did not show a difference in burst strength with the addition of tannic acid. In contrast, soybean and potato pectin films demonstrated significantly lower burst strength (p<0.001). FIG. 7B is a group of images showing fracture patterns reflected the cohesion measurements. Citrus pectin showed little effect of the tannic acid; however, both soybean and potato pectin demonstrated brittle films. FIG. 7C is a graph quantifying the adhesion force of citrus pectin films. The adhesive force between citrus pectin films was increased with increasing concentrations of tannic acid (p<0.05). FIG. 7D is a graph quantifying the extensibility of citrus pectin films. The extensibility of the pectin films was lower, but not statistically significant, with increasing tannic acid concentrations (p>0.05). Error bars=1 SD.

### DETAILED DESCRIPTION

The compositions described herein include bioadhesive polymer (e.g., pectin-based) film compositions. In some examples, the compositions described herein are used for sealing a wound (e.g., a wound in a visceral organ). Some embodiments of the film compositions and methods described herein may provide one or more of the following advantages.

Certain embodiments of the present disclosure include bioadhesive, pectin-based polymer film compositions, some of which are referred to herein as pectin films. Some embodiments described herein may provide bioadhesive polymer films that may have tunable degradability and controlled release of a drug loaded into the film. For example, in some embodiments, the degradability (e.g., facial erosion) of the bioadhesive polymer films described herein can be controlled by varying the amount of tannic acid in the film composition. For example, the degradation half-life of the films can be increased by increasing the amount of tannic acid in the film compositions. By controlling the degradation half-life of the films, the release of a loaded drug (e.g., a hydrophilic drug or a hydrophobic drug) can also be controlled. Thus, in some embodiments, the compositions and methods of the disclosure may provide a flexible sealant that can be optimized for various tissue types, tissue injuries, and delivery cargoes.

In addition, some embodiments described herein may provide a bioabsorbable sealant. For example, in contrast to sutures, the bioadhesive polymer compositions described herein are bioabsorbable so there is no need for postoperative removal of any compositions.

The film compositions and methods of the present disclosure provide a wound sealant that is simple, efficient, and effective. For example, in some embodiments, the films described herein do not require the addition of curing agents (e.g., photocrosslinkers) and additional steps (e.g., exposure to light) to initiate adhesion onto a tissue or to seal a wound in a tissue. The bioadhesive, pectin-based films of the disclosure adhere to a tissue within seconds.

For example, the methods of the disclosure can require an application routine that is reminiscent of the application of pressure sensitive adhesives (e.g., Scotch® tape). In other words, adhesion of the bioadhesive polymer films described herein can develop in seconds. Thus, in some embodiments, the film compositions and methods of the disclosure provide a user (e.g., a clinician) with an efficient method to easily seal a wound in a tissue that requires no additional steps other than contacting the bioadhesive film(s) with the tissue of interest. For example, in some embodiments, the methods described do not require a user to mix various components. In some embodiments, the bioadhesive polymer compositions of the disclosure do not have a limited time window within which a user must apply the composition to a tissue before an undesirable change to the composition occurs (e.g., unwanted solidification, unwanted separation of components, or the like). Thus, some embodiments described herein may provide methods of sealing a wound that are simple and time-efficient.

In another example, in some embodiments, the films described herein can have sufficient adhesive strength to adhere to a tissue (e.g., a visceral tissue and/or an ocular tissue) for a prolonged period of time without falling off. In some embodiments, the films of the disclosure, once adhered to an injured tissue, can have a pressure resistance sufficient to remain adhered to the injured tissue through cyclical fluctuations (e.g., increase and decrease of pressure in a tissue or organ due to inspiration or expiration). In some embodiments, the films of the disclosure can help prevent wound leakage after surgery. The bioadhesive polymer compositions, e.g., pectin tape, can be capable of limiting wound leakage despite wide ranges in pressures.

Furthermore, the bioadhesive polymer compositions described herein are biocompatible and safe. For example, the bioadhesive polymer compositions include HMP, which has been widely recognized as a harmless food additive in North America and Europe. In the United States, pectin is affirmed GRAS (Generally Recognized as Safe) as defined in the Code of Federal Regulations (21CFR184.1588).

### Film Compositions

The present disclosure features bioadhesive polymer compositions that can include one or more films comprising high-methoxyl pectin (HMP), tannic acid, and water.

Pectins are a family of plant cell wall polysaccharides and/or glycan domains that consist mainly of esterified D-galacturonic acid residues in (1→4) chains. Pectins differ from typical pressure sensitive adhesives, as they do not bind to most non-biologic compounds. However, they selectively and strongly bind to the mesothelial glycocalyx, which may be likely the result of a mechanism of interdiffusion or interpenetration, e.g., the entanglement of branched chain polysaccharides based on chemical bonds and weak chemical interactions.

Pectins can vary in molecular weight, cross-linking density (determined by multi-angle laser light scattering), and chemical groups (e.g., hydroxyl, amine, sulfur and carboxyl groups). The polysaccharides that make up pectin are generally grouped into three major types: homogalacturonan (HG), rhamnogalacturonan I (RG-I), and the substituted galacturonan rhamnogalacturonan II (RG-II). Some plant cell walls also contain additional substituted galacturonans, known as apiogalacturonan (AGA) and xylogalacturonan (XGA). Thus, pectins are often defined by their source, e.g., citrus pectin (which was used in the examples described herein). The most common categories of pectin vary with respect to amidation and methoxylation; however, all pectins appear to be biocompatible. When exposed to calcium, pectin forms egg box-like structures that facilitate the immobilization of substances within the gel structure.

The pectins in the film compositions described herein are preferably high-methoxyl pectins (HMP), which can be obtained commercially (e.g., from Cargill, Minneapolis, Minn., USA). The proportion of galacturonic acid residues in the methyl ester form determines the degree of methoxylation or methyl esterification. The proportion of galacturonic acid residues found in pectin in the methyl ester form determines the degree of methoxylation. HMPs are defined herein as those pectins with a degree of methoxylation greater than about 50%; low-methoxyl pectins (LMP) are defined herein as those pectins with a degree of methoxylation of less than about 50%.

Disclosed herein, in certain embodiments, are bioadhesive polymer compositions including a film including a polymer comprising HMP, tannic acid, and water. In some embodiments, the film compositions include a citrus HMP having a degree of methoxylation or methyl esterification greater than about 50%. In some embodiments, the film compositions include HMP having a degree of methoxylation or methyl esterification ranging from about 50% to about 100% (e.g., about 50% to about 60%, about 50% to about 70%, about 50% to about 80%, about 50% to about 90%, about 60% to about 70%, about 60% to about 80%, about 60% to about 90%, about 60% to about 100%, about 70% to about 80%, about 70% to about 90%, about 70% to about 100%, about 80% to about 90%, or about 80% to about 100%). In some embodiments, the film compositions include HMP having a degree of methoxylation or methyl esterification of about 50%. In some embodiments, the film compositions include HMP that contains one or more of HG, a partially methyl esterified polymer of galacturonic acid (e.g., (1-4)-α-D-galacturonic acid (GalA)), RG-I, RG-II, AGA, and XGA.

In some embodiments, the film compositions include HMP at an initial concentration ranging from of about 1% (w/w) to about 10% (w/w) (e.g., about 1% (w/w) to about 3% (w/w), about 1% (w/w) to about 4% (w/w), about 1% (w/w) to about 5% (w/w), about 1% (w/w) to about 6% (w/w), about 1% (w/w) to about 7% (w/w), about 1% (w/w) to about 8% (w/w), about 1% (w/w) to about 9% (w/w), about 2% (w/w) to about 3% (w/w), about 2% (w/w) to about 4% (w/w), about 2% (w/w) to about 5% (w/w), about 2% (w/w) to about 6% (w/w), about 2% (w/w) to about 7% (w/w), about 2% (w/w) to about 8% (w/w), about 2% (w/w) to about 9% (w/w), about 3% (w/w) to about 4% (w/w), about 3% (w/w) to about 5% (w/w), about 3% (w/w) to about 6% (w/w), about 3% (w/w) to about 7% (w/w), about 3% (w/w) to about 8% (w/w), about 3% (w/w) to about 9% (w/w), or about 3% (w/w) to about 10% (w/w)). In some embodiments, the film compositions include HMP at an initial concentration of about 3% (w/w).

As used herein, the term “initial concentration” of HMP refers to the concentration of HMP at the time when HMP is dissolved in water, thereby forming a viscous solution. For example, the initial concentration of HMP is the concentration of HMP prior to the step of adding tannic acid and evaporating the water from the viscous solution (e.g., prior to inducing the gel transition and polymerization of the viscous solution). The steps of preparing the films of the disclosure are disclosed elsewhere herein (e.g., in Example 1).

The loss of water alone from a dispersed solution of HMP can lead to the initial polymerization of the pectin. This so-called “gel transition” is associated with a discrete change in the physical properties of the pectin from a viscous liquid to a soft and rubbery gel. The ongoing loss of water from the pectin gel leads to a second discrete step, so called “glass transition,” associated with a change in the physical properties of the pectin from soft and rubbery to hard and brittle. The compositions described herein include a film in a gel phase or a glass phase. In some embodiments, when in a gel phase, the film is soft, pliable, flexible, rubbery, moldable, or any combination thereof. In some embodiments, when in a glass phase, the film is rigid and brittle. In some embodiments, the bioadhesive film is a moldable film.

As described above, the films of the disclosure include tannic acid. Tannic acid is a specific form of tannin, a type of polyphenol, having a weak acidity due to the numerous phenol groups in its structure. Without being bound to any theory, it is believed that tannic acid is a potential pectin chain crosslinker. In some embodiments, the films disclosed herein have a controlled degradation rate that is dependent upon the amount of tannic acid present in the film composition. For example, in some embodiments, the degradation half-life of the films increases with an increasing amount of tannic acid present in the film composition. In some embodiments, the loading of tannic acid into the films of the disclosure exhibits a dose-dependent decrease in pectin erosion.

In some embodiments, the film compositions include tannic acid at an initial concentration ranging from about 0.2% (w/w) to about 2% (w/w) or more (e.g., about 0.2% (w/w) to about 0.25% (w/w), about 0.2% (w/w) to about 0.27% (w/w), about 0.2% (w/w) to about 0.3% (w/w), about 0.2% (w/w) to about 0.4% (w/w), about 0.2% (w/w) to about 0.5% (w/w), about 0.2% (w/w) to about 0.75% (w/w), about 0.2% (w/w) to about 0.81% (w/w), about 0.2% (w/w) to about 1% (w/w), about 0.2% (w/w) to about 1.25% (w/w), about 0.2% (w/w) to about 1.35% (w/w), about 0.2% (w/w) to about 1.50% (w/w), about 0.2% (w/w) to about 1.62% (w/w), about 0.2% (w/w) to about 1.75% (w/w), about 0.2% (w/w) to about 1.89% (w/w), about 0.3% (w/w) to about 0.4% (w/w), about 0.3% (w/w) to about 0.5% (w/w), about 0.3% (w/w) to about 0.75% (w/w), about 0.3% (w/w) to about 1% (w/w), about 0.3% (w/w) to about 1.25% (w/w), about 0.3% (w/w) to about 1.50% (w/w), about 0.3% (w/w) to about 1.75% (w/w), about 0.3% (w/w) to about 2% (w/w), about 0.4% (w/w) to about 0.5% (w/w), about 0.4% (w/w) to about 0.75% (w/w), about 0.4% (w/w) to about 1% (w/w), about 0.4% (w/w) to about 1.25% (w/w), about 0.4% (w/w) to about 1.50% (w/w), about 0.4% (w/w) to about 1.75% (w/w), about 0.4% (w/w) to about 2% (w/w), about 0.5% (w/w) to about 0.75% (w/w), about 0.5% (w/w) to about 1% (w/w), about 0.5% (w/w) to about 1.25% (w/w), about 0.5% (w/w) to about 1.50% (w/w), about 0.5% (w/w) to about 1.75% (w/w), about 0.5% (w/w) to about 2% (w/w), about 0.6% (w/w) to about 0.75% (w/w), about 0.6% (w/w) to about 1% (w/w), about 0.6% (w/w) to about 1.25% (w/w), about 0.6% (w/w) to about 1.50% (w/w), about 0.6% (w/w) to about 1.75% (w/w), about 0.6% (w/w) to about 2% (w/w), about 0.7% (w/w) to about 0.75% (w/w), about 0.7% (w/w) to about 1% (w/w), about 0.7% (w/w) to about 1.25% (w/w), about 0.7% (w/w) to about 1.50% (w/w), about 0.7% (w/w) to about 1.75% (w/w), about 0.7% (w/w) to about 2% (w/w), about 0.8% (w/w) to about 1% (w/w), about 0.8% (w/w) to about 1.25% (w/w), about 0.8% (w/w) to about 1.50% (w/w), about 0.8% (w/w) to about 1.75% (w/w), about 0.8% (w/w) to about 2% (w/w), about 0.9% (w/w) to about 1% (w/w), about 0.9% (w/w) to about 1.25% (w/w), about 0.9% (w/w) to about 1.50% (w/w), about 0.9% (w/w) to about 1.75% (w/w), about 0.9% (w/w) to about 2% (w/w), about 1% (w/w) to about 1.25% (w/w), about 1% (w/w) to about 1.50% (w/w), about 1% (w/w) to about 1.75% (w/w), about 1% (w/w) to about 2% (w/w), about 1.25% (w/w) to about 1.50% (w/w), about 1.25% (w/w) to about 1.75% (w/w), about 1.25% (w/w) to about 2% (w/w), about 1.50% (w/w) to about 1.75% (w/w), about 1.50% (w/w) to about 2% (w/w), or about 1.75% (w/w) to about 2% (w/w)).

In some embodiments, the film compositions include tannic acid at an initial concentration ranging from about 0.2% (w/w) to about 5% (w/w). In some embodiments, the film compositions include tannic acid at an initial concentration ranging from about 0.2% (w/w) to about 10% (w/w). In some embodiments, the film compositions include tannic acid at an initial concentration ranging from about 5% (w/w) to about 10% (w/w).

As used herein, the term “initial concentration” of tannic acid refers to the concentration of tannic acid when HMP and tannic acid are dissolved in water, and the film composition is in a viscous solution state. For example, the initial concentration of tannic acid is the concentration of tannic acid prior to the step of evaporating the water from the viscous solution that includes HMP and tannic acid (e.g., prior to inducing the gel transition and polymerization of the viscous solution includes HMP and tannic acid).

In some embodiments, the film compositions include water at an initial concentration ranging from about 1% (w/w) to about 10% (w/w) (e.g., about 1% (w/w) to about 5% (w/w), about 1% (w/w) to about 6% (w/w), about 1% (w/w) to about 7% (w/w), about 1% (w/w) to about 8% (w/w), about 1% (w/w) to about 9% (w/w), about 2% (w/w) to about 5% (w/w), about 2% (w/w) to about 6% (w/w), about 2% (w/w) to about 7% (w/w), about 2% (w/w) to about 8% (w/w), about 2% (w/w) to about 9% (w/w), about 2% (w/w) to about 10% (w/w), about 3% (w/w) to about 5% (w/w), about 3% (w/w) to about 6% (w/w), about 3% (w/w) to about 7% (w/w), about 3% (w/w) to about 8% (w/w), about 3% (w/w) to about 9% (w/w), about 3% (w/w) to about 10% (w/w), about 4% (w/w) to about 5% (w/w), about 4% (w/w) to about 6% (w/w), about 4% (w/w) to about 7% (w/w), about 4% (w/w) to about 8% (w/w), about 4% (w/w) to about 9% (w/w), about 4% (w/w) to about 10% (w/w), about 5% (w/w) to about 6% (w/w), about 5% (w/w) to about 7% (w/w), about 5% (w/w) to about 8% (w/w), about 5% (w/w) to about 9% (w/w), or about 5% (w/w) to about 10% (w/w)). In some embodiments, the film compositions include water at an initial concentration ranging of about 5% (w/w).

As used herein, the term “initial concentration” of water refers to the concentration of water when HMP and tannic acid are dissolved in water, and the film composition is in a viscous solution state. For example, the initial concentration of water is the concentration of water prior to the step of evaporating the water from the viscous solution that includes HMP and tannic acid (e.g., prior to inducing the gel transition and polymerization of the viscous solution includes HMP and tannic acid).

In some embodiments, the bioadhesive films include one or more active agents. In some embodiments, the films exhibit a controlled release of one or more active agents that is determined by the degradation rate of the film or the facial erosion rate of the film, once adhered to a tissue. In some embodiments, the degradation rate or the facial erosion rate is determined by the amount of tannic acid present in the films. For example, in some embodiments, the degradation rate of the film or the facial erosion rate decreases with an increased concentration of tannic acid in the film.

In some embodiments, the bioadhesive film has a degradation half-life or a facial erosion half-life ranging from about 20 hours (h) to about 200 h (e.g., about 20 h to about 50 h, about 20 h to about 75 h, about 20 h to about 80 h, about 20 h to about 100 h, about 20 h to about 150 h, about 20 h to about 200 h, about 50 h to about 75 h, about 50 h to about 80 h, about 50 h to about 100 h, about 50 h to about 150 h, about 50 h to about 200 h, about 75 h to about 80 h, about 75 h to about 100 h, about 75 h to about 150 h, about 75 h to about 200 h, about 75 h to about 100 h, about 75 h to about 150 h, about 75 h to about 200 h, about 80 h to about 100 h, about 80 h to about 150 h, about 80 h to about 200 h, about 100 h to about 150 h, about 100 h to about 200 h, or about 150 h to about 200 h.) In some embodiments, the bioadhesive film has a degradation half-life of about 20 h. In some embodiments, the bioadhesive film has a degradation half-life of about 69 h. In some embodiments, the bioadhesive film has a degradation half-life of about 79 h. In some embodiments, the bioadhesive film has a degradation half-life of about 99 h. In some embodiments, the bioadhesive film has a degradation half-life of about 154 h.

In some embodiments, the one or more active agents can include a hydrophobic drug, a hydrophilic drug, or both. For example, the one or more active agents can include one or more anti-bacterial agents or anti-fungal agents. Non-limiting examples of anti-bacterial agents and anti-fungal agents include ciprofloxacin, levofloxacin, doxycycline hyclate, ofloxacin, erythromycin, cefazolin, vancomycin, gentamycin, tobramycin, ceftazidime, gatifloxacin, amphotericin, voriconazole, natamycin, bacitracin, besifloxacin, moxifloxacin, and tobramycin.

In some embodiments, the one or more active agents can include one or more anti-inflammatory agents. Non-limiting examples of anti-inflammatory agents include corticosteroids, loteprednol etabonate, prednisolone acetate, dexamethasone, lifitegrast, cyclosporine, bromfenac, nepafenac, ketorolac, diclofenac, suprofen, flurbiprofen, aspirin, ibuprofen, ketoprofen, non-steroidal anti-inflammatory drugs, or any combination thereof.

In some embodiments, the one or more active agents can include one or more growth factors, e.g., one or more of transforming growth factor alpha (TGF-α) and TGF-β, tumor necrosis factor-alpha (TNF-α), vascular endothelial growth factor (VEGF), leukemia inhibitory factor (LIF), interleukins such as IL-1 through IL-7, colony-stimulating factors such as macrophage colony-stimulating factor (m-CSF), granulocyte colony-stimulating factor (G-CSF), and granulocyte macrophage colony-stimulating factor (GM-CSF), fibroblast growth factor (FGF), epidermal growth factor (EGF), insulin-like growth factor, connective tissue growth factor (CTGF), hepatocyte growth factor (HGF), angiopoietin-1-4, and platelet-derived growth factor (PDGF). In some embodiments, the one or more films comprise the active agents.

In some embodiments, the one or more active agents can include one or more of heparin, tissue plasminogen activator (tPA). In some embodiments, the one or more active agents can include hormones, cytokines, osteogenic factors, chemotactic factors, proteins and peptides that contain an arginine-glycine-aspartate (“RGD”) motif, analgesics, anesthetics, a vasoconstrictor, a clotting factor, a chemotherapy agent, an immunotherapy agent, or any combination thereof. In some embodiments, the vasoconstrictor includes one or more of norepinephrine, epinephrine, phenylpropanolamine, dopamine, metaraminol, methoxamine, ephedrine, and propylhexedrine. In some embodiments, the clotting factor includes fibrillar collagen, thrombin, fibrin, or any combination thereof. In some embodiments, the films of the disclosure do not include carboxymethylcellulose (CMC).

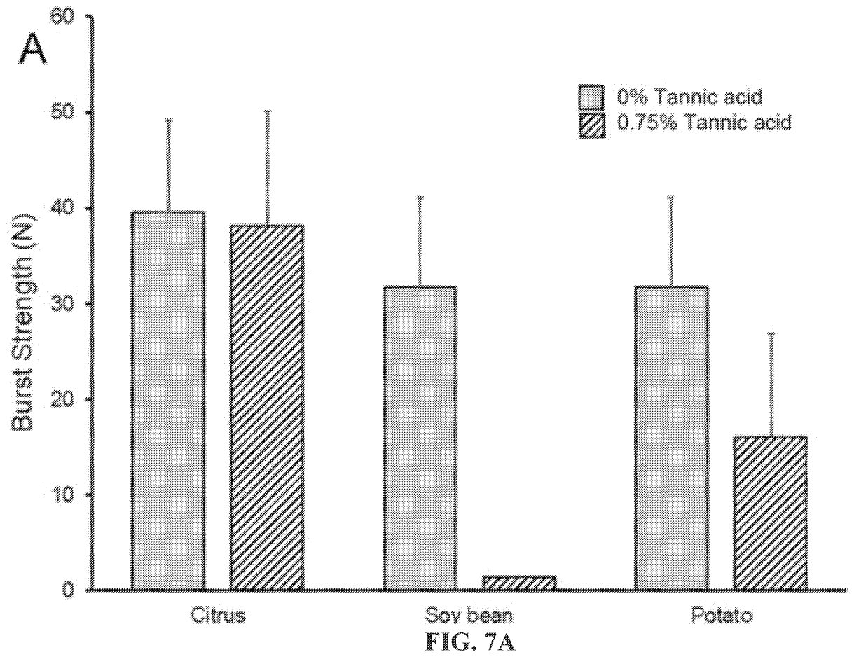

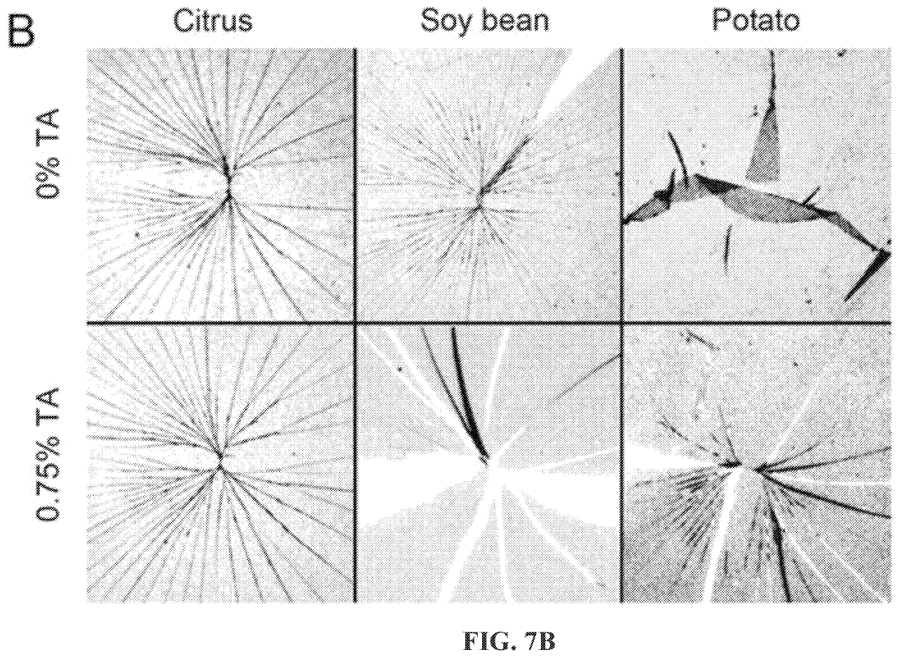

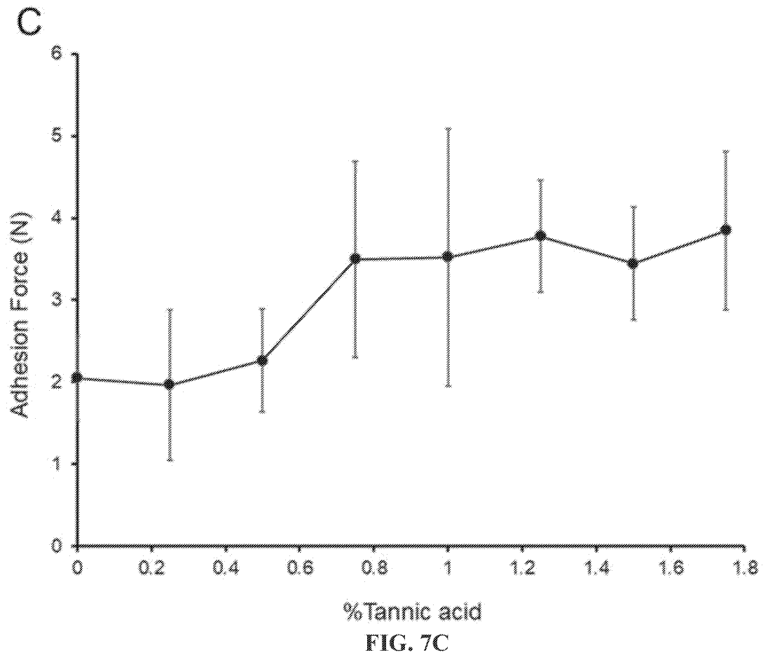

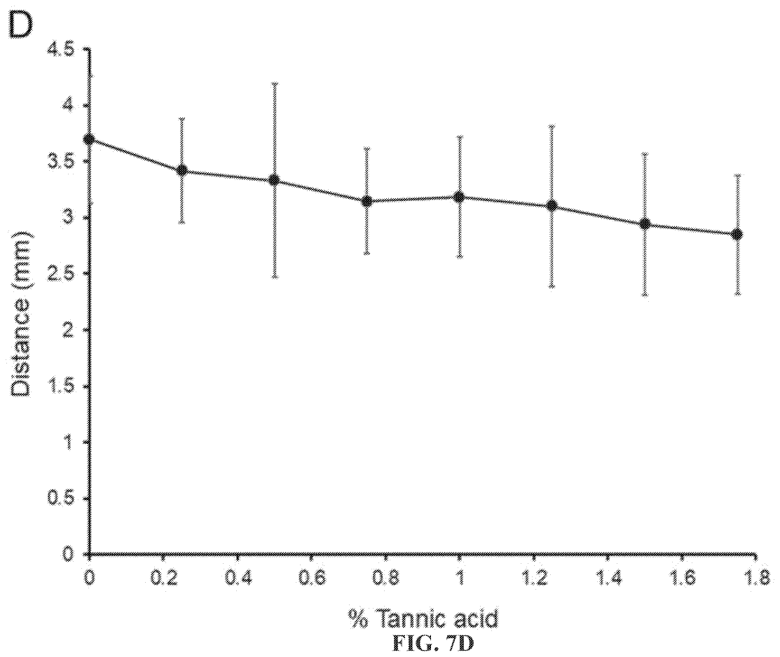

In some embodiments, tannic acid has no detectable impact on the physical properties of citrus pectin films including adhesivity and cohesion. In contrast, and as described in Example 2 and as shown in FIGS. 7A and 7B, tannic acid weakens the burst strength and cohesion of pectins derived from soybean and potato sources. In some embodiments, the adhesive force between a first bioadhesive film and a second bioadhesive film increases with increasing concentrations of tannic acid. In some embodiments, the adhesive force between a bioadhesive film and a tissue (e.g., a mesothelial tissue or an optical tissue) increases with increasing concentrations of tannic acid.

In some embodiments, the adhesive force of the bioadhesive films of the disclosure ranges from about 1 newton (N) to about 5 N (e.g., about 1 N to about 2 N, about 1 N to about 3 N, about 1 N to about 4 N, about 2 N to about 3 N, about 2 N to about 4 N, about 2 N to about 5 N, about 3 N to about 4 N, about 3 N to about 5 N, or about 4 N to about 5 N). In some embodiments, the adhesive force of the bioadhesive films of the disclosure is about 3 N.

In some embodiments, tannic acid has no significant effect on the burst strength of the bioadhesive films of the disclosure. In some embodiments, the burst strength of the bioadhesive films of the disclosure ranges from about 30 newton (N) to about 50 N (e.g., about 30 N to about 35 N, about 30 N to about 40 N, about 30 N to about 45 N, about 30 N to about 49 N, about 35 N to about 40 N, about 35 N to about 45 N, about 35 N to about 50 N, about 40 N to about 45 N, about 40 N to about 50 N, or about 45 N to about 50 N). In some embodiments, the burst strength of the bioadhesive films of the disclosure is about 40 N.

In some embodiments, tannic acid has no significant effect on the extensibility of the bioadhesive films of the disclosure. In some embodiments, the extensibility of the bioadhesive films of the disclosure decreases with an increasing concentration of tannic acid in the films. In some embodiments, the extensibility of the bioadhesive films of the disclosure ranges from about 2.5 millimeters (mm) to about 4.5 mm (e.g., about 2.5 mm to about 3 mm, about 2.5 mm to about 3.5 mm, about 2.5 mm to about 4 mm, about 3 mm to about 3.5 mm, about 3 mm to about 4 mm, about 3 mm to about 4.5 mm, about 3 mm to about 5 mm, about 3.5 mm to about 4 mm, about 3.5 mm to about 4.5 mm, about 3.5 mm to about 5 mm, about 4 mm to about 4.5 mm, about 4 mm to about 5 mm, or about 4.5 mm to about 5 mm). In some embodiments, the extensibility of the bioadhesive films of the disclosure is about 3 mm.

In some embodiments, the bioadhesive polymer film has a thickness ranging from about 40 μm to about 200 μm (e.g., about 40 μm to about 50 μm, about 40 μm to about 60 μm, about 40 μm to about 70 μm, about 40 μm to about 80 μm, about 40 μm to about 90 μm, about 40 μm to about 100 μm, about 40 μm to about 110 μm, about 40 μm to about 120 μm, about 40 μm to about 130 μm, about 40 μm to about 140 μm, about 40 μm to about 150 μm, about 40 μm to about 160 μm, about 40 μm to about 170 μm, about 40 μm to about 180 μm, about 40 μm to about 190 μm, about 40 μm to about 199 μm, about 50 μm to about 60 μm, about 50 μm to about 70 μm, about 50 μm to about 80 μm, about 50 μm to about 90 μm, about 50 μm to about 100 μm, about 50 μm to about 110 μm, about 50 μm to about 120 μm, about 50 μm to about 130 μm, about 50 μm to about 140 μm, about 50 μm to about 150 μm, about 50 μm to about 160 μm, about 50 μm to about 170 μm, about 50 μm to about 180 μm, about 50 μm to about 190 μm, about 50 μm to about 200 μm, about 60 μm to about 70 μm, about 60 μm to about 80 μm, about 60 μm to about 90 μm, about 60 μm to about 100 μm, about 60 μm to about 110 μm, about 60 μm to about 120 μm, about 60 μm to about 130 μm, about 60 μm to about 140 μm, about 60 μm to about 150 μm, about 60 μm to about 160 μm, about 60 μm to about 170 μm, about 60 μm to about 180 μm, about 60 μm to about 190 μm, about 60 μm to about 200 μm, about 70 μm to about 80 μm, about 70 μm to about 90 μm, about 70 μm to about 100 μm, about 70 μm to about 110 μm, about 70 μm to about 120 μm, about 70 μm to about 130 μm, about 70 μm to about 140 μm, about 70 μm to about 150 μm, about 70 μm to about 160 μm, about 70 μm to about 170 μm, about 70 μm to about 180 μm, about 70 μm to about 190 μm, about 70 μm to about 200 μm, about 80 μm to about 90 μm, about 80 μm to about 100 μm, about 80 μm to about 110 μm, about 80 μm to about 120 μm, about 80 μm to about 130 μm, about 80 μm to about 140 μm, about 80 μm to about 150 μm, about 80 μm to about 160 μm, about 80 μm to about 170 μm, about 80 μm to about 180 μm, about 80 μm to about 190 μm, or about 80 μm to about 200 μm). In some embodiments, the bioadhesive polymer composition comprises a film having a thickness of about 40 μm to about 50 μm.

In some embodiments, the bioadhesive polymer composition further includes a pharmaceutically acceptable carrier. As used herein, the expression “pharmaceutically acceptable carrier” refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation and is compatible with administration to a subject, for example a human. It must also be suitable for use in contact with any tissues or organs with which it may come in contact, meaning that it must not carry a risk of toxicity, irritation, allergic response, immunogenicity, or any other complication that excessively outweighs its therapeutic benefits. Examples of pharmaceutically acceptable carriers include, but are not limited to, a solvent or dispersing medium containing, for example, water, pH buffered solutions (e.g., phosphate buffered saline (PBS), HEPES, TES, MOPS, etc.), isotonic saline, Ringer's solution, polyol (for example, glycerol, propylene glycol, liquid polyethylene glycol, and the like), alginic acid, ethyl alcohol, and suitable mixtures thereof. In some embodiments, the pharmaceutically acceptable carrier can be a pH buffered solution (e.g. PBS).

In some embodiments, the pharmaceutically acceptable carrier is a topical carrier. In some embodiments, the bioadhesive polymer film is formulated for topical use. In some embodiments, the film is topically administered to a tissue (e.g., an ocular tissue or a mesothelial tissue) of a patient. In some embodiments, the film can be applied to a tissue (e.g., an ocular tissue or a mesothelial tissue) to seal an injury (e.g., an ocular injury or an injury to a mesothelial tissue).

### Methods of Preparation and Testing

Certain embodiments of the disclosure include methods of preparing a bioadhesive, pectin-based polymer film. In some embodiments, the methods include providing a film comprising a polymer comprising HMP, tannic acid, and water. For example, the film can include any of compositions described elsewhere herein.

In some embodiments, the film can be prepared by the methods described in Example 1. For example, the first film can be prepared by gradually dissolving the polymer (e.g., HMP) powder in water at a temperature of about 25 °C. For example, the HMP powder can be dissolved by a step-wise increase in added water to avoid undissolved powder. In some embodiments, the HMP powder is dissolved to a mixing concentration of about 3% (w/w) (e.g., about 3 g of HMP powder is dissolved in about 100 g of water). In some embodiments, the HMP powder is dissolved to a mixing concentration of about 1% (w/w) to about 3% (w/w). Next, the methods include mixing at a high shear (e.g., mixing at about 10,000 revolutions per minute (RPM)) to achieve fluidization and complete dissolution of HMP.

In some embodiments, tannic acid in a powder form is loaded into the HMP viscous solution. Next, the methods include mixing at a high shear (e.g., mixing at about 10,000 RPM) to achieve fluidization and complete dissolution of the tannic acid. In another example, the tannic acid in a powder form can be added to the HMP solution before complete dissolution of the HMP. In some embodiments, no exogenous heat is used to dissolve the polymer (e.g., HMP) and/or the tannic acid.

Next, the dissolved polymer solution can be transferred to one or more molds and “cured” (e.g., via a controlled evaporation of water) in an environment having a controlled humidity. In some embodiments, the controlled humidity environment is a low humidity environment (e.g., less than about 20% relative humidity) that enables the progressive loss of water content of the polymer solution in the mold. In some embodiments, the thickness of the films can be controlled by the dimensions of the mold in which the polymer solution is poured into and cured.

Pectin can demonstrate striking and reversible changes in their physical properties in the presence of even trace amounts of water. The loss of water alone from a dispersed solution of HMP can lead to the initial polymerization of the pectin. This so-called “gel transition” is associated with a discrete change in the physical properties of the HMP from a viscous liquid to a soft and rubbery gel. The ongoing loss of water from the HMP gel leads to a second discrete step, so-called “glass transition,” associated with a change in the physical properties of the HMP from soft and rubbery to hard and brittle.

In some embodiments, the methods include progressively evaporating the water in each film until the film is in a gel phase or a glass phase. In some embodiments, the film has a final water content (e.g., after the step of progressively evaporating the water) ranging from about 37% to about 43% (w/w) once it is in the gel phase. In some embodiments, the film has a final water content ranging from about 37% (w/w) to about 43% (w/w) (e.g., about 37% (w/w) to about 38% (w/w), about 37% (w/w) to about 39% (w/w), about 37% (w/w) to about 40% (w/w), about 37% (w/w) to about 41% (w/w), about 37% (w/w) to about 42% (w/w), about 37% (w/w) to about 43% (w/w), about 38% (w/w) to about 39% (w/w), about 38% (w/w) to about 40% (w/w), about 38% (w/w) to about 41% (w/w), about 38% (w/w) to about 42% (w/w), about 38% (w/w) to about 43% (w/w), about 39% (w/w) to about 40% (w/w), about 39% (w/w) to about 41% (w/w), about 39% (w/w) to about 42% (w/w), about 39% (w/w) to about 43% (w/w), about 40% (w/w) to about 41% (w/w), about 40% (w/w) to about 42% (w/w), about 40% (w/w) to about 43% (w/w), about 41% (w/w) to about 42% (w/w), about 41% (w/w) to about 43% (w/w), or about 42% (w/w) to about 43% (w/w)). In some embodiments, the film has a final water content of about 40% (w/w).

In some embodiments, the film has a final water content ranging from about 9% to about 13% (w/w) once it is in the glass phase. In some embodiments, the film has a final water content ranging from about 9% (w/w) to about 13% (w/w) (e.g., about 9% (w/w) to about 10% (w/w), about 9% (w/w) to about 11% (w/w), about 9% (w/w) to about 12% (w/w), about 10% (w/w) to about 11% (w/w), about 10% (w/w) to about 12% (w/w), about 10% (w/w) to about 13% (w/w), about 11% (w/w) to about 12% (w/w), about 11% (w/w) to about 13% (w/w), or about 12% (w/w) to about 13% (w/w)). In some embodiments, the film has a final water content of about 10% (w/w).

As used herein, the term “final water content” of the film refers to the water content of the film after the water in the film composition is progressively evaporated (e.g., after the film is poured in a mold) to reach a desired concentration. The progressive evaporation of water can occur at the time the film composition is a viscous solution, thereby inducing the polymerization and gel transition that changes the viscous solution into a soft, rubbery film. Additionally, the progressive evaporation of water can occur at the time the film composition is a gel phase, thereby inducing the glass transition that changes the soft, rubbery film into a hard and brittle film.

In some embodiments, the films do not include conjoined films (e.g., a first bioadhesive, pectin-based film in a gel or glass phase adhered to a second bioadhesive, pectin-based film in a gel or glass phase).

Another aspect of the present disclosure is directed to a method for assessing facial erosion of a film (e.g., of the bioadhesive films of the disclosure). The assessment of facial erosion in bioadhesive films may provide a useful method for identifying modifiers of citrus pectin biodegradation. Distinct from immersion testing, which may be typically used to assess surface or bulk erosion in films, assessing the rate of facial erosion may facilitate the characterization of tannic acid as a modifier of pectin biodegradation. In some embodiments, the dynamics of pectin biodegradation in a setting of facial exposure can be assessed by using a microfluidics system to enable multiple parallel comparisons of pectin films exposed to facial erosion; e.g., exposure limited to a water interface.

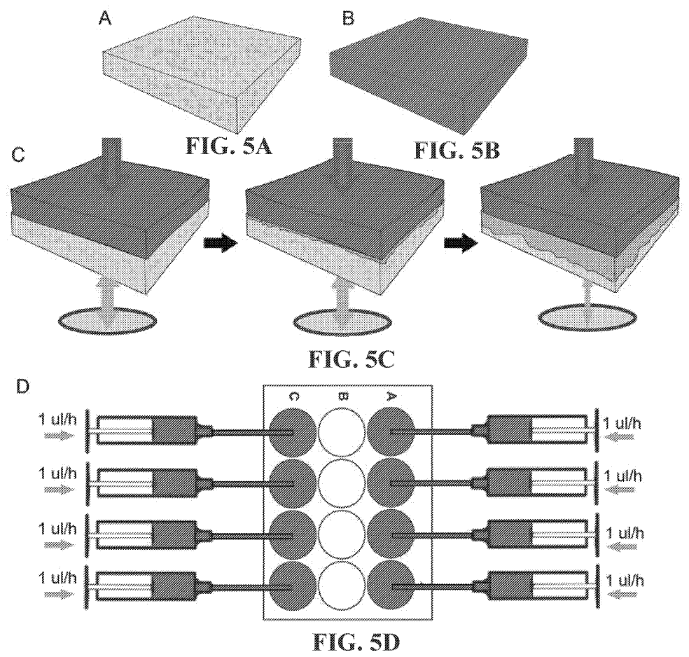

In some embodiments, the facial erosion of the film can be assessed by the methods described in Examples 1 and 2. For example, a fluorescent tracer (e.g., fluorescein) can be loaded into the pectin solution during high-shear mixing. Next, as shown in FIGS. 5A-5D, a microfluidics system including the fluorescein-embedded pectin polymers overlaid with a porous black matrix (to simulate attachment to an organ) can be perfused with a quencher agent that quenches fluorescein (e.g., trypan blue). Serial microfluorimetry measurements of the fluorescein tracer embedded in the films can then be recorded from the pectin aspect of the interface in order to assess degradation or facial erosion kinetics. In some embodiments, the microfluidic system includes 8 parallel syringe pumps perfusing the interface of the films. In some embodiments, the perfusion rate is 1 μL/h.

### Methods of Treatment

In some embodiments, the films of the disclosure may facilitate the sealing of a visceral organ (e.g., lung, liver, bowel, heart, or any combination thereof) wound of a subject upon application and adhesion. In some embodiments, the films of the disclosure may facilitate the sealing of a wound of a mesothelial tissue of a subject upon application and adhesion. In some embodiments, the films of the disclosure may facilitate the sealing of a wound of a tissue having a glycocalyx surface (e.g., lung, liver, bowel, heart, eye, or any combination thereof) upon application and adhesion. In some embodiments, the HMP chains of the compositions exhibit entanglement with the glycocalyx of a tissue upon contact, thereby resulting in adhesion of the compositions to the tissue.

In some embodiments, the films of the disclosure may facilitate the sealing and/or treating of an ocular injury in an eye of a subject upon application and adhesion. For example, the films of the disclosure can be contacted with an ocular surface, can subsequently adhere to the ocular surface, and can seal an ocular injury. In some embodiments, the methods of sealing and/or treating an ocular injury include providing a bioadhesive polymer composition that is a single film. In some embodiments, the methods of sealing and/or treating an ocular injury include providing a bioadhesive polymer composition that includes two or more films adhered to each other.

### Mesothelial Tissue Injuries

In some embodiments, the bioadhesive, pectin-based polymer film binds to a mesothelial tissue. In some embodiments, the mesothelial tissue includes one or more mesothelial tissues from a visceral organ. In some embodiments, the visceral organ is an internal organ of the abdominal, thoracic, and pelvic cavities. Non-limiting examples of visceral organs include one or more of a lung, a heart, a pancreas, a liver, a gall bladder, a spleen, a kidney, a stomach, a colon, a small intestine, a large intestine, and a bladder.

In some embodiments, the methods of covering and sealing a wound in an injured mesothelial tissue of a subject can include providing a bioadhesive film comprising a polymer comprising HMP, tannic acid, and water (e.g., any of the bioadhesive films of the disclosure). Next, the methods can include contacting the wound of the subject with the bioadhesive polymer film. In some embodiments, the methods include applying pressure to the bioadhesive polymer film once it comes in contact with the wound or mesothelial tissue. In some embodiments, the pressure applied to the bioadhesive polymer film is a light pressure (e.g., about the same amount of pressure applied when adhering a piece of Scotch® tape to a paper). In some embodiments, the methods do not require the step of applying pressure to the bioadhesive film once it comes in contact with the wound. In some embodiments, the pressure is applied for about 10 seconds(s) or less (e.g., about 1 s to about 2 s, about 1 s to about 3 s, about 1 s to about 4 s, about 1 s to about 5 s, about 1 s to about 6 s, about 1 s to about 7 s, about 1 s to about 8 s, about 1 s to about 9 s, about 1 s to about 10 s, or about 5 s to about 10 s). In some embodiments, the bioadhesive film reaches 80% of maximal adhesion to the wound or mesothelial tissue within at least about 5 seconds of contact, thereby sealing the wound in the mesothelial tissue of the subject. In some embodiments, the bioadhesive film adheres to the wound of the subject with an adhesion strength of at least about 3 N. In some embodiments, the bioadhesive film remains localized over the incision, injury, and/or laceration to seal the wound and form a surface barrier. In some embodiments, the bioadhesive film is a biocompatible and adhesive sealant on the ocular surface.

### Ocular Injuries

The present disclosure presents methods and film compositions for treating ocular injuries (e.g., ocular surface injuries) in an eye of a subject. In some embodiments, the ocular injury is a corneal injury. In some embodiments, the ocular injury is a corneal incision. In some embodiments, the ocular injury is an injury or trauma resulting from an ocular surgery. In some embodiments, the ocular surgery is cataract surgery. In some embodiments, the compositions of the disclosure are used in post-surgical care. For example, the compositions can be administered to a patient after an ocular surgery to deliver a therapeutic agent (e.g., an anti-inflammatory agent or an antibiotic) that can be prescribed to minimize recovery time, prevent and/or treat inflammation caused by the surgical procedure, prevent and/or treat an ocular infection caused by the surgical procedure, or any combination thereof.

Ocular surface injuries can include conjunctival laceration, corneal perforation, scleral perforation, incisions due to ocular surgery (e.g., cataract surgery) or any combination thereof. In some embodiments, the ocular surface injury is a corneal or scleral injury. Conjunctival laceration can occur following blunt or penetrating trauma. Conjunctival lacerations can be associated with chemosis and subconjunctival hemorrhage. In such cases, it is important to rule out underlying scleral perforation. The fundus should be examined for any retinal tear or intraocular foreign body. An ultrasound can be done for the posterior segment evaluation.

Corneal lacerations and perforations represent approximately 1 in 10 of ocular traumatic injuries presenting in an emergency medical setting. Corneal lacerations and perforations can include partial thickness lacerations and full thickness lacerations. In addition, adnexal injuries, scleral perforation, or a combination thereof can be involved with corneal laceration and perforations. The standard of care for a corneal perforation includes the removal of any contaminants in the wound area, repair of the tear, and maintenance of the watertight integrity of the ocular globe. Corneal perforation can also be associated with or caused by insertion of a foreign body. In some embodiments, the corneal injury is a corneal full-thickness laceration or a corneal full-thickness perforation. In some embodiments, the ocular surface injury is a full-thickness laceration or a full-thickness perforation. In some embodiments, the ocular surface injury is a full-thickness laceration or surgical incision or a full-thickness perforation. For example, the majority of ocular surgeries that require entry into the eye (e.g., cataract surgery) involve a full-thickness incision through the cornea or sclera. Current management protocols for full thickness lacerations including scleral wounds often require the use of sutures.

The compositions of the disclosure can be used to treat ocular incisions or cuts or injuries having a length of less than about 1 mm to about 10 mm. In some embodiments, the compositions of the present disclosure can be used in the closure of full-thickness ocular defects and lacerations and in controlled and long-term drug elution. In some embodiments, indications can include post-operative applications of the biomaterial for drug elution in addition of closure of corneal ulcers, defects and perforations caused by a wide array of insults. The compositions of the disclosure can be applied both under “normal” (e.g., in-the-office or operating room) settings, or under emergency “in-in-field” settings. Various providers, physicians, and, in select cases, physician assistants and paramedics (e.g., in the combat theater) can apply the compositions described herein to seal the eye and elute drug(s) to heal defects. The compositions described herein can circumvent many cases of transplants and patch grafts for corneal melts and defects.

### EXAMPLES

Certain embodiments of the present disclosure are further described in the following examples, which do not limit the scope of any embodiments described in the claims.

### Example 1—Synthesis and Physical Characterization of Pectin Films Pectin

Citrus pectins were obtained from a commercial source (Cargill, Minneapolis, MN, USA). The citrus pectins were high-methoxyl pectins (HMP); that is, pectin polymer with greater than 50% degree of methoxylation. Soybean and potato pectins were obtained from Megazyme (Bray, Ireland). The pectin powder was stored in low humidity at 25 °C prior to use.

### Pectin Dissolution in Water

The pectin powder was dissolved by a step-wise increase in added water to avoid undissolved pectin. Swelling and softening of the particles were followed by fluidization and dissolution. The complete dissolution of the pectin was achieved by a high-shear 10,000 revolutions per minute (RPM) rotor-stator mixer (L5M-A, Silverson, East Longmeadow, MA, USA). To ensure reproducible mixing, viscosity monitoring was performed with a digital tachometer and ammeter (DataLogger, Silverson). The dissolved pectin was poured into a standard mold for further studies.

### Tracer Embedding

The fluorescent tracer fluorescein sodium (C₂₀H₁₀Na₂O₃; MW 376.27), obtained in powder form (Sigma-Aldrich, St. Louis, MO, USA), was dissolved in water to create a 1 mg/mL concentrated stock solution of fluorescein. The fluorescein tracer was loaded into the pectin during high-shear mixing.

### Fluorescent Measurement

The fluorescein tracer embedded in the pectin film was measured with a CytoFluor® 4000 Fluorescence Measurement System (Millipore, Bedford, MA, USA). The CytoFluor® 4000 is a computer-controlled multi-well fluorescence scanning device with a tungsten halogen lamp and a broadband interference filter for fluorescein: excitation 450±50 nm and emission 530±25 nm. Intensity readings were obtained from the bottom of the microfluidics plate as relative fluorescence unites (RFU). Background fluorescence (medium alone) was measured separately for each plate and subtracted from all readings. The data were exported to the Microsoft Excel 360 (Redmond, WA, USA) spreadsheet for data analysis. Each experiment was performed in triplicate or quadruplicate. The standard deviation (SD) of replicate measures was uniformly less than 30% of the mean.

### Quenching Agent

Trypan blue solution (C₃₄H₂₄N₆Na₄O₁₄S₄; MW 960) was obtained in a 0.4% solution in 0.81% NaCl and 0.06% KPO₄ (Sigma). The trypan blue was used at a final concentration of 0.04% (w/v) or as indicated.

### Tannic Acid

The polyphenolic compound tannic acid (C₇₆H₅₂O₄₆; MW 1701.20) was obtained from commercial sources as an undissolved powder (Sigma-Aldrich, St. Louis, MO, USA). The tannic acid was loaded into the citrus pectin at concentrations of 0%, 0.27%, 0.81%, 1.35%, 1.62%, and 1.89% w/w in water, using a high-shear mixer.

### Microperfusion

The microperfusion system was controlled with New Era Pump Systems NE-1800 multi-channel programmable syringe pump (Farmingdale, NY, USA). In most experiments, 1 mL syringes (BH Supplies, Jackson, NJ, USA) loaded with 0.4% trypan blue dye perfused the matrix interface through 0.46 mm-diameter polyethylene tubing (Instech, Plymouth Meeting, PA, USA) at 1 μL/h. The circular 14 mm-diameter tracer embedded pectin films were placed in the bottom of a 24-multiwell clear flat bottom cell culture plate (Falcon, Corning, NY, USA) and overlaid with a circular (12 mm-diameter) black 6 μm-pore matrix (Thomas Scientific, Swedesboro, NJ, USA) loaded with 17 μL of 0.4% trypan blue prior to constant rate perfusion.

### Adhesion Testing

Polymer-polymer adhesion experiments were performed with a custom fixture designed for the TA-XT plus with a 50 kg load cell (Stable Micro Systems, 110 Godalming, Surrey, UK). The fixture was composed of a 25 mm-diameter flat-ended acrylic cylindrical probe and a flat acrylic fixture surface. The 20 mm-diameter films, affixed to both surfaces, were hydrated with 7 μL of distilled water. The cylindrical probe compressed the polymers at a force of 5 N and a development time of 10 s followed by probe withdrawal at 0.2 mm/s. Data was acquired at a rate of 500 points per second (pps).

### Cohesion Testing

Fracture mechanics were performed as follows. Briefly, the biopolymers were subjected to a controlled uniaxial load normal to the plane of the polymer film. The load was applied with a 5 mm stainless-steel spherical probe mounted to a TA-XT plus (Stable Micro Systems) with a 50 kg load cell. The stainless-steel ball was positioned centrally over the biopolymer. The probe compressed the biopolymers at a test speed of 2 mm/s until fracture. The fracture force, distance, and time were recorded at 500 pps.

### Example 2—Biodegradation of Pectin Films

### Fluorescein Tracer

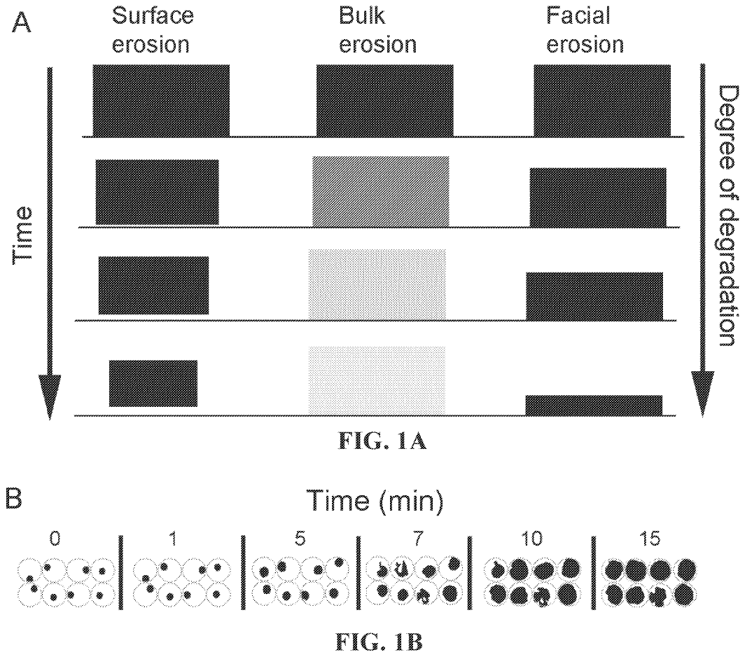

Biodegradation decreases polymer mass as a function of time. In addition to the commonly recognized mechanisms of surface and bulk erosion, there are selected physiologic environments that reflect facial erosion (FIG. 1A). A common method for evaluating surface and bulk erosion is water immersion. When pectin film biodegradation was assessed by water immersion, there was a rapid swelling and dissolution of the films (FIG. 1B). The rapidity of degradation precluded effective analysis of the process.

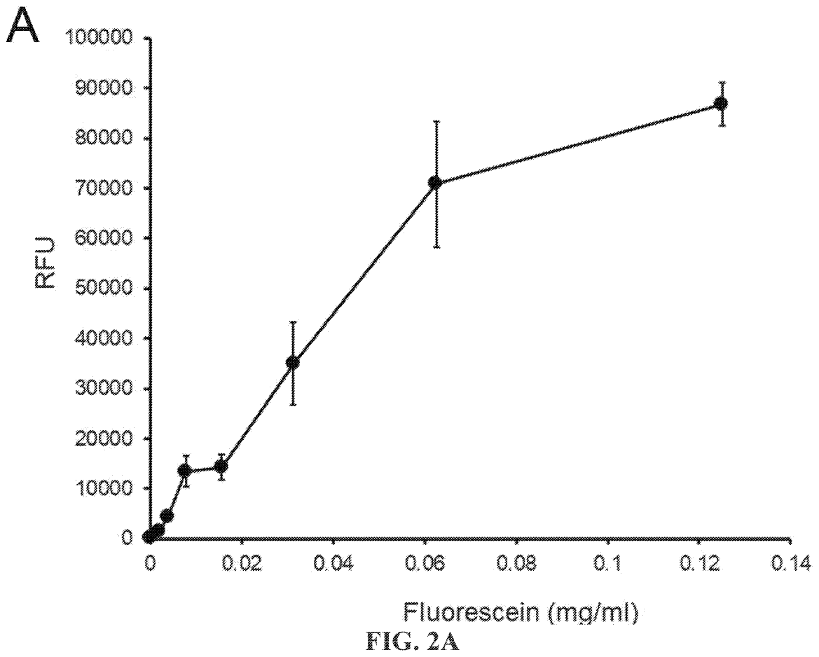

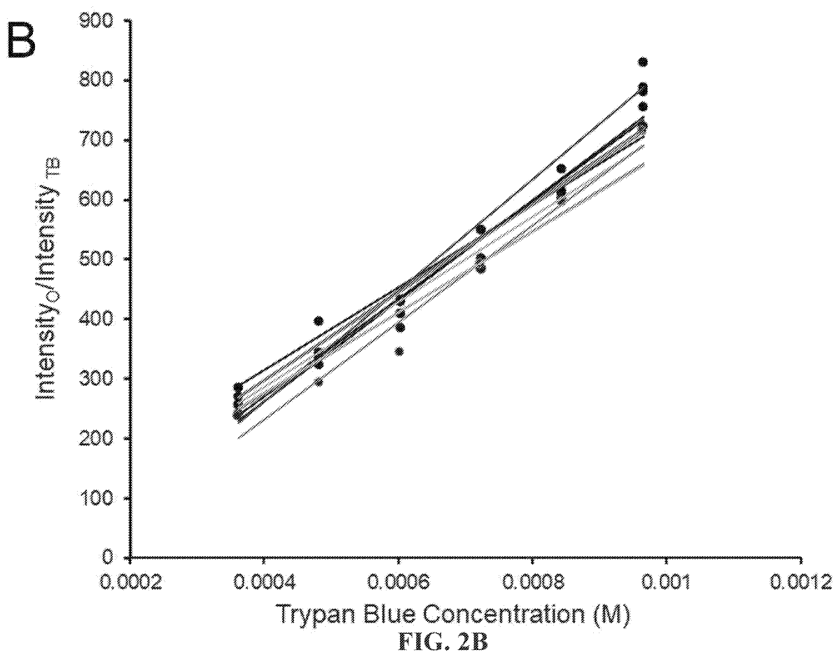

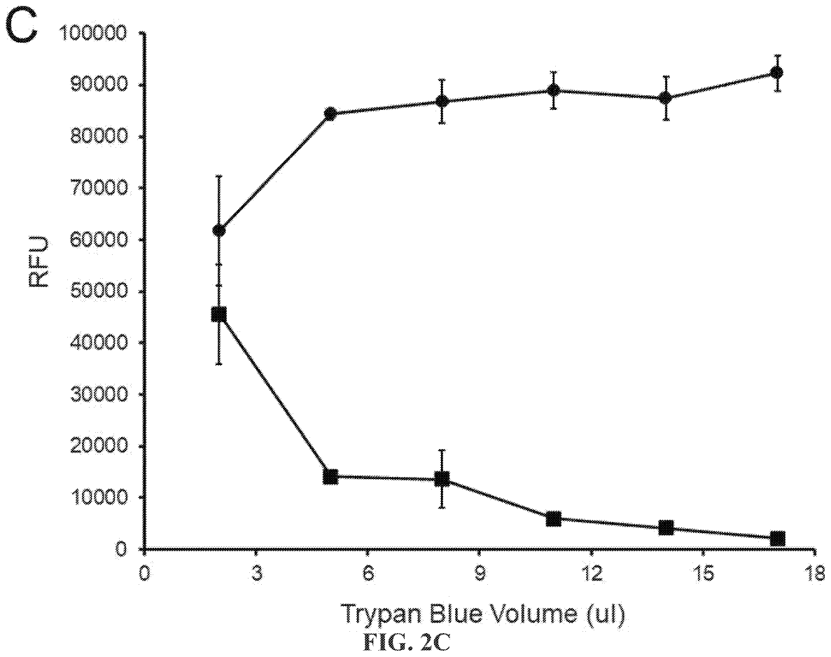

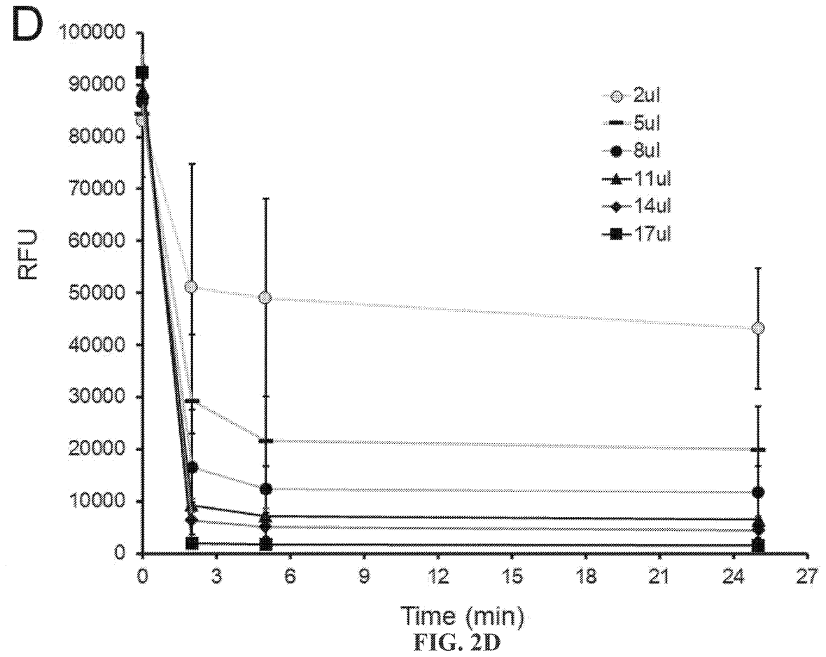

To quantitatively assess facial erosion of pectin films, fluorochromes were evaluated for their potential utility as tracers for pectin biodegradation. Fluorescein was readily detected by microfluorimetry and was linear over a wide concentration range (FIG. 2A). The fluorescein molecules demonstrated no evidence of biochemical reactivity with pectin films and were efficiently quenched with trypan blue (Stern-Vollmer constant 7.15×10⁵ M⁻¹) (FIG. 2B). Using standard 0.4% trypan blue solutions, 4 μL of a standard concentration of fluorescein (125 μg/mL) was effectively quenched by 5 μL of trypan blue (FIG. 2C). The quenching was stable over time (FIG. 2D).

### Free Volume Embedding

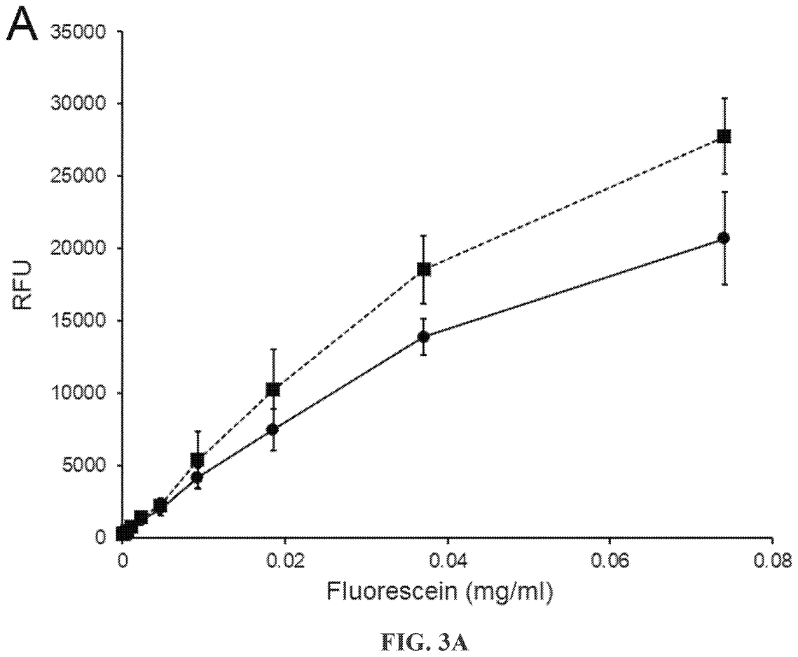

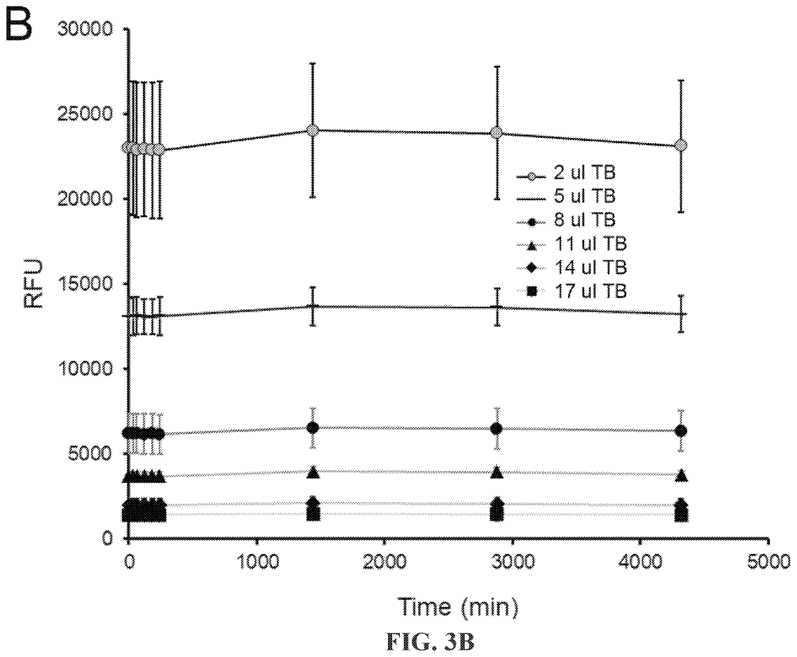

To determine the effect of pectin embedding on the fluorescein signal, increasing concentrations of fluorescein were embedded into the pectin films. Fluorimetry demonstrated a largely linear increase over a significant range of concentrations (FIG. 3A). Solubilization of the pectin film produced a modest, but proportional, increase in fluorescence intensity—a change likely reflecting concentration-dependent auto-quenching (FIG. 3A). A range of fluorescein concentrations were stable in the embedded films for more than 72 h (FIG. 3B).

### Matrix Interface

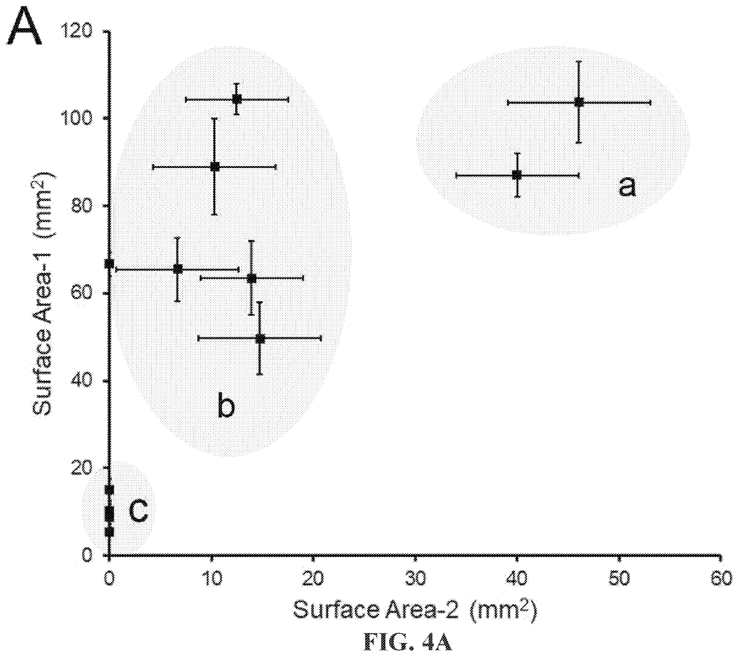

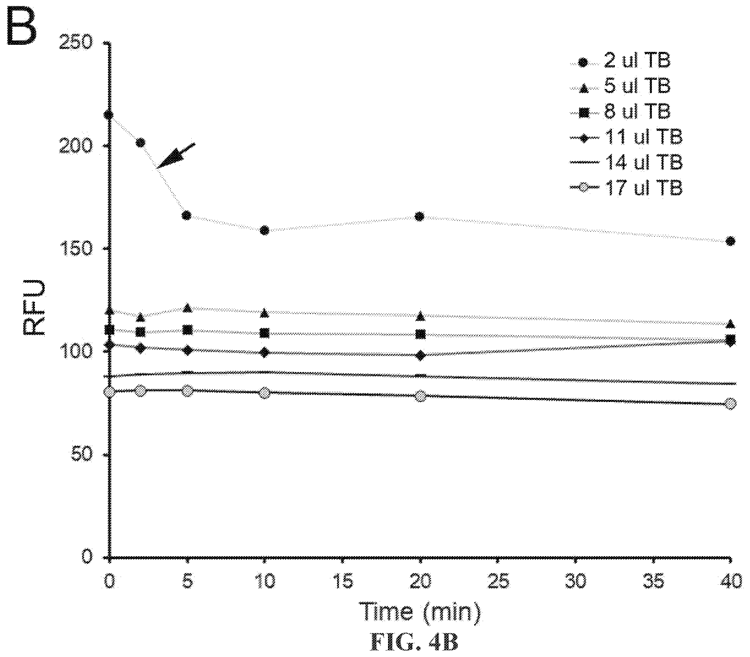

To obtain a wettable and erosive interface, a variety of porous matrices were screened for their ability to disperse the microfluidic perfusate. Most of the matrices were cellulose-based (cellulose acetate and nitrocellulose) filters, but a variety of nylon and synthetic membranes were screened as well (FIG. 4A). To evaluate lateral and penetrating diffusion, double-thickness matrices were tested. The surface area of the superficial matrix (Surface Area-1) was compared to the surface area of the deeper layer (Surface Area-2). The ideal matrix maximized lateral diffusion while minimizing penetrating diffusion. The matrices with both lateral diffusion and penetration (FIG. 4A (a)) provided a less well-developed interface when compared to synthetic membranes (FIG. 4B (b)) or cellulose-based filters (FIG. 4A (c)). Synthetic 6 μm-pore membranes were found to provide an optimal interface. To minimize light reflectance artifact, a black membrane was used.

### Microfluidics of Facial Erosion

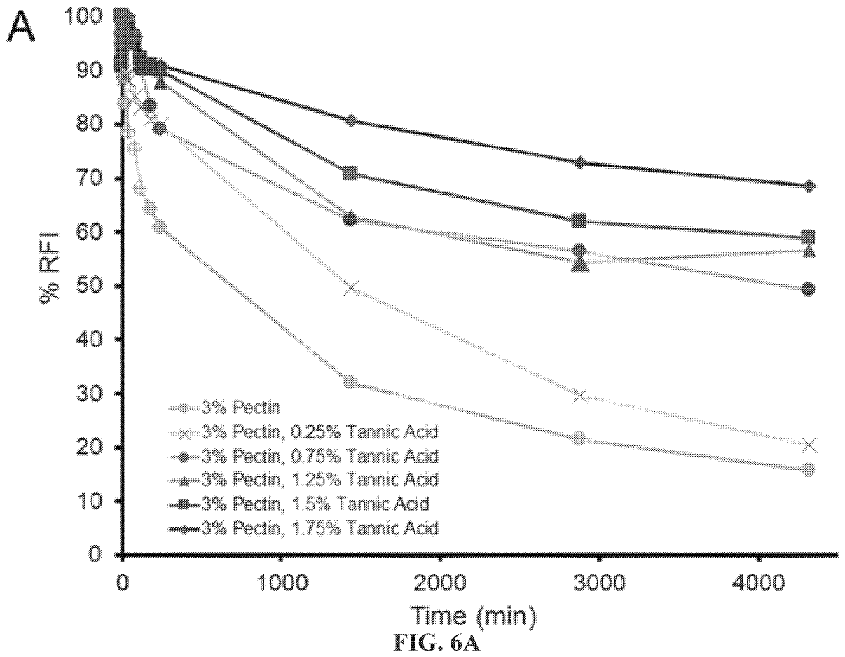

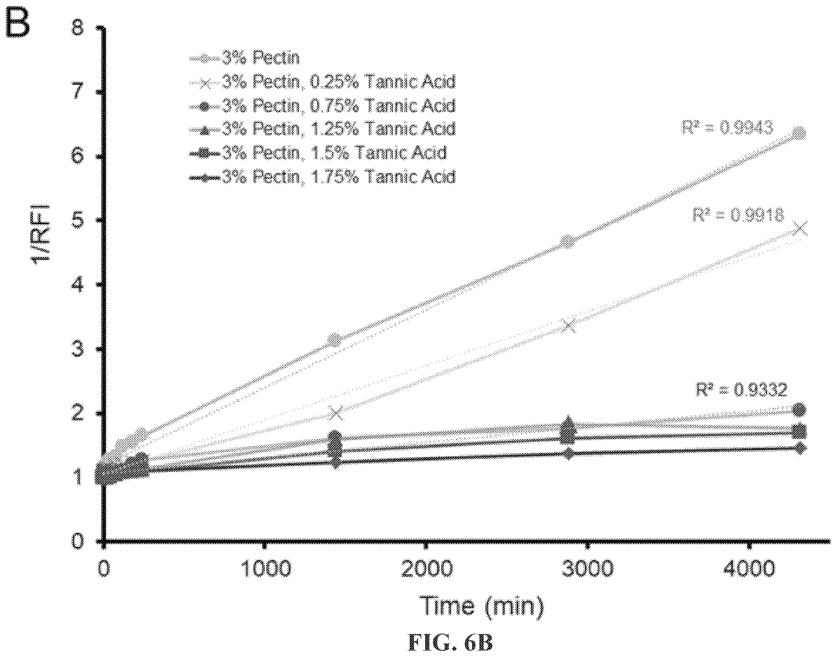

The microfluidics system consisted of fluorescein-embedded pectin polymers (FIG. 5A) overlaid with a porous black matrix (FIG. 5B) perfused with 0.4% trypan blue (FIG. 5C). Serial microfluorimetry measurements were recorded from the pectin aspect of the interface. The system used 8 parallel syringe pumps perfusing the interface at 1 μL/h (FIG. 5D). When citrus pectin films were studied, there was nonlinear erosion of the pectin film (FIG. 6A, orange); the tracer was reduced by 75% in 28 h. To demonstrate the potential effect of a crosslinker on pectin erosion, the molecule tannic acid was added to the film. Tannic acid is an important member of the natural polyphenol family. When added to pectin films, tannic acid demonstrated a dose-dependent decrease in pectin erosion. When plotted as the inverse of residual fluorescence (1/RFU), the pectin tracer demonstrated linear kinetics (FIG. 6B).

The effect of tannic acid on the physical properties varied with the source of pectin. Citrus high-methoxyl pectin, the pectin source used in the previous experiments, demonstrated no significant change in pectin film burst strength. In contrast, tannic acid weakened the burst strength of pectins derived from soybean and potato sources (FIG. 7A). Consistent with the burst strength findings, the fracture patterns of citrus pectin were similar with and without tannic acid. Soybean and potato films demonstrated significant changes in fracture patterns with the addition of tannic acid (FIG. 7B). Finally, tannic acid did not diminish pectin-pectin adhesivity (FIG. 7C) nor did it significantly impact citrus pectin extensibility (FIG. 7D).

### Example 3—Tannic Acid as a Modifier of Pectin Biodegradation

In this report, a microfluidics system to quantitatively evaluate the facial erosion of pectin films was developed. The system had 3 components: (1) a fluorescein tracer embedded in the pectin free volume, (2) a wettable porous matrix interface, and (3) a microfluidics perfusion system that facilitated multiple parallel comparisons. Signal isolation was enhanced with the incorporation of trypan blue as a quencher of released fluorescence. The utility of this system to evaluate facial erosion was demonstrated with the study of tannic acid. A potential pectin chain crosslinker, tannic acid demonstrated a highly significant dose-dependent modification of pectin erosion.

There are multiple definitions of biodegradability. As used herein, the term biodegradability refers to the degradation of polymers subjected to a physiologic environment where the polymers are broken down into nontoxic fragments (monomers) and eliminated over time. Although physiologic environments offer many potential biochemical reactions, it is assumed that the reaction with water is the dominant degradation process for pectin in vivo. Hydrogels are degraded by hydrolysis of the polymer backbone and/or hydrolysis of the cross-links. A common approach to testing the rate of biopolymer hydrolysis is water immersion. Immersion testing simply involves immersion of the film in water followed by quantifying the polymer mass loss as a function of time. Although the testing is straightforward, the rapid rate of physical dissolution (minutes) complicates attempts to evaluate the primary physical process or the effectiveness of biodurability modifiers. Moreover, the rapid rate of dissolution observed in immersion testing is inconsistent with biological observations. In contrast to the rapid degradation in immersion testing, the estimated half-life of pectin films is 7 days in vivo.

To provide a more quantitative measure of biodegradation as well as a more physiologic microenvironment, a microfluidics assay designed to explore the facial erosion of pectin films was developed. To facilitate wetting and avoid flow artifacts at the pectin surface, a variety of porous matrices to create the facial interface was screened. Most of the screened membrane filters were composed of cellulose nitrate, cellulose acetate, or less refined pulp filters. The optimal synthetic porous membrane had a pore size that optimized lateral diffusion while minimizing penetrating flow artifact.

Critical to the highly quantitative measure of facial erosion was the use of fluorescein tracers embedded in the pectin films. The use of fluorescein tracers reflected the presence of a free volume within the pectin polymer. The free volume represents the unoccupied volume of the pectin film. Since this volume cannot be measured and theoretical models are uncertain, fluorescein was empirically embedded into the pectin film. Informed by experience using pectin as a drug delivery vehicle, the physical properties of the tracer-embedded pectin films were tested. The fluorescein tracer provided a strong fluorescence signal with no discernable effect on the pectin films' physical properties. The pectin films with embedded fluorescein tracer demonstrated nearly identical physical properties to pectin films without the tracer. Notably, the burst strength, an objective measure of the films' cohesive properties, was unchanged with the addition of the fluorescein tracer.

To improve signal isolation of the embedded tracer, trypan blue was used to quench the released fluorescein. A textile dye and vital stain, trypan blue is an effective fluorescence quencher. Trypan blue had no measurable biochemical effect on the pec-tin polymer. At a standard 0.4% w/v concentration, stock trypan blue solutions provided both fluorescence quenching and water erosion.

The advantage of protracted degradation is the opportunity to quantitatively evaluate modifiers of pectin biodurability. An effective modifier was tannic acid. Tannic acid is a polyphenolic compound with multiple potential interactions with the heteropolysaccharide pectin including hydrogen bonding, hydrophobic interactions, covalent bonding, and electrostatic interactions. The most likely interaction, however, is hydrogen bonding. Tannic acid's abundant phenolic groups serve as a hydrogen donor to link with hydrogen acceptors to form a stronger hydrogen bond. In this case, tannic acid may function to increase chain interactions and decrease solubility by substituting for the water-pectin chain interactions. The size of the tannic acid molecule also suggests the potential for crosslinking between polymer chains. Regardless of the mechanism, the results of our microfluidics system suggest that tannic acid will have a significant impact on pectin durability in vivo.

Finally, the release of the embedded fluorescent tracer has obvious implications for drug delivery. Pectin is an extraordinary bioadhesive with the ability to adhere to the surface of virtually all visceral organs. In addition to targeting visceral organs, the data here indicate that the pectin matrix can effectively deliver substantial concentrations of embedded drugs. The ultimate in situ biodegradation of the pectin is an additional attractive feature of pectin embedded drug delivery.

### Other Embodiments

It is to be understood that while certain embodiments have been described within the detailed description, the present disclosure is intended to illustrate and not limit the scope of any embodiment defined by the scope of the appended claims. Other aspects, advantages, and modifications are within the scope of the appended claims.

1.-33. (canceled)

34. A bioadhesive, pectin-based polymer film comprising:
a high-methoxyl pectin (HMP);
tannic acid; and
water,
wherein the pectin-based polymer film has a controlled degradation rate that is dependent on an amount of tannic acid present in the pectin-based polymer film.

35. The bioadhesive, pectin-based polymer film of claim 34, wherein HMP is present at an initial concentration of about 1% w/w to about 10% w/w; or wherein HMP is present at an initial concentration of about 3% w/w; and wherein tannic acid is present at an initial concentration of about 0.2% w/w to about 2% w/w.

36. The bioadhesive, pectin-based polymer film of claim 34, wherein water is present at an initial concentration of about 1% w/w to about 10% w/w; or wherein water is present at an initial concentration of about 5% w/w.

37. The bioadhesive, pectin-based polymer film of claim 34, wherein the pectin-based polymer film has a degradation half-life ranging from about 20 hours to about 200 hours; and wherein the degradation is facial erosion.

38. The bioadhesive, pectin-based polymer film of claim 34, wherein the pectin-based polymer film has a thickness of about 40 to 200 microns; or wherein the pectin-based polymer film has a thickness of about 40 to 50 microns.

39. The bioadhesive, pectin-based polymer film of claim 34, wherein the pectin-based polymer film comprises one or more active agents; and wherein the one or more active agents comprise an anti-thrombotic agent, an anti-inflammatory agent, a hormone, a cytokine, an osteogenic factor, a chemotactic factor, a protein or peptide that contain an arginine-glycine-aspartate (“RGD”) motif, an analgesic, an anesthetic, a vasoconstrictor, a clotting factor, a chemotherapy agent, an immunotherapy agent, a growth factor, or a combination thereof.

40. The bioadhesive, pectin-based polymer film of claim 39, wherein the anti-thrombotic agent is heparin, tissue plasminogen activator (tPA), or a combination thereof; wherein the anti-inflammatory agent is aspirin, ibuprofen, ketoprofen, a non-steroidal anti-inflammatory drug, or a combination of thereof; and wherein the vasoconstrictor is norepinephrine, epinephrine, phenylpropanolamine, dopamine, metaraminol, methoxamine, ephedrine, propylhexedrine, fibrillar collagen, thrombin, fibrin, or a combination thereof.

41. The bioadhesive, pectin-based polymer film of claim 39, wherein the growth factor comprises transforming growth factor alpha (TGF-α), TGF-β, tumor necrosis factor-alpha (TNF-α), vascular endothelial growth factor (VEGF), leukemia inhibitory factor (LIF), interleukins, IL-1 through IL-7, colony-stimulating factors, macrophage colony-stimulating factor (m-CSF), granulocyte colony-stimulating factor (G-CSF), granulocyte macrophage colony-stimulating factor (GM-CSF), fibroblast growth factor (FGF), epidermal growth factor (EGF), insulin-like growth factor, connective tissue growth factor (CTGF), hepatocyte growth factor (HGF), angiopoietin-1-4, platelet-derived growth factor (PDGF), or any combination of distinct growth factors thereof.

42. The bioadhesive, pectin-based polymer film of claim 34, wherein the HMP is a citrus HMP having a degree of methoxylation greater than about 50%; and wherein the pectin-based polymer film is a moldable film.

43. The bioadhesive, pectin-based polymer film of claim 34, wherein the bioadhesive, pectin-based polymer film binds to a mesothelial tissue; and wherein the mesothelial tissue comprises a mesothelial tissue from a visceral organ.

44. A method of covering and sealing a wound in an injured mesothelial tissue of a subject in need thereof, the method comprising:
providing a bioadhesive pectin-based polymer film comprising: a high-methoxyl pectin (HMP), tannic acid, and water;
contacting the wound with the bioadhesive pectin-based polymer film; and
applying pressure to the bioadhesive pectin-based polymer film, thereby sealing the wound in the injured mesothelial tissue of the subject,
wherein the bioadhesive pectin-based polymer film has a controlled degradation rate that is dependent on an amount of tannic acid present in the bioadhesive pectin-based polymer film.

45. The method of claim 44, wherein the HMP is present at an initial concentration of about 1% w/w to about 10% w/w; or wherein the HMP is present at an initial concentration of about 3% w/w.

46. The method of claim 44, wherein the tannic acid is present at an initial concentration of about 0.2% w/w to about 2% w/w.

47. The method of any one of claim 44, wherein the water is present at an initial concentration of about 1% w/w to about 10% w/w; or wherein the water is present at an initial concentration of about 5% w/w.

48. The method of any one of claim 44, wherein the bioadhesive pectin-based polymer film has a degradation half-life ranging from about 20 hours to about 200 hours; and wherein the degradation is facial erosion.

49. The method of any one of claim 44, wherein the bioadhesive pectin-based polymer film has a thickness of about 40 to 3,000 microns.

50. The method of any one of claim 44, wherein the bioadhesive pectin-based polymer film comprises one or more active agents.

51. The method of claim 50, wherein the one or more active agents comprise an anti-thrombotic agent, an anti-inflammatory agent, a hormone, a cytokine, an osteogenic factor, a chemotactic factor, a protein or peptide that contain an arginine-glycine-aspartate (“RGD”) motif, an analgesic, an anesthetic, a vasoconstrictor, a clotting factor, a chemotherapy agent, an immunotherapy agent, a growth factor, or any combination of distinct active agents thereof.

52. The method of claim 44, wherein the HMP is a citrus HMP having a degree of methoxylation greater than about 50%.

53. The method of any one of claim 44, wherein the injured mesothelial tissue comprises a mesothelial tissue from a visceral organ.
